(19) United States
(12) Patent Application Publication
(10) Pub. No.: US 2026/0124031 A1
(43) Pub. Date: May 7, 2026

(54) SUTURE FIXATION SYSTEMS FOR LIGAMENT RECONSTRUCTION

(71) Applicant: Amir JAMALI, Oakland, CA (US)

(72) Inventor: Amir JAMALI, Oakland, CA (US)

(21) Appl. No.: 18/937,112

(22) Filed: Nov. 5, 2024

(51) Int. Cl. A61F 2/08

(52) U.S. Cl. CPC A61F 2/0811; A61F 2002/0817; A61F 2002/0852; A61F 2002/0882

(57) ABSTRACT

A suture fixation system for ligament reconstruction may include a suture lock, which may include a proximal end including a first portion, a distal end including a second portion that may be smaller than the first portion and configured to be received in a bone tunnel, a ledge portion configured to inhibit the first portion from entering the bone tunnel, and suture apertures extending from the distal end to the proximal end and configured to allow the sutures to pass through the suture lock. The system may further include a fastener including a fastener head and configured to engage the suture lock. With the sutures applying tension to the ligament graft, the fastener may be configured to securably pinch the sutures between the fastener head and the suture lock to inhibit sliding of the sutures relative to the suture lock to maintain the tension of the ligament graft.

### TECHNICAL FIELD

The present disclosure relates to systems and methods for use in orthopedic surgery. More specifically, the present disclosure relates to suture fixation systems for ligament reconstruction.

### BACKGROUND

Ligaments in the knee are typically divided into those that are on the outside of the joint (extraarticular) such as the medial collateral ligament (MCL) and the lateral collateral ligament (LCL) or on the inside of the joint (intraarticular) such as the anterior cruciate ligament (ACL) or posterior cruciate ligament (PCL). The extraarticular ligaments typically can heal with nonsurgical treatment occasionally requiring a brace. This is due to the nature of their anatomy being superimposed on the joint capsule. The joint capsule provides a template for the ligament to heal from its origin to its insertion through the elaboration of scar tissue. The situation is more complex in the case of intraarticular ligaments.

These typically do not heal without surgical intervention since the two ends have no soft tissue connection upon which scar tissue can be laid. As a result, the intraarticular ligaments usually require reconstruction with a separate source of tissue. The tissue used for reconstruction of ligaments can be either from the patient's own body, termed an autograft, from a donor individual, termed an allograft, or from a synthetic source such as a collagen bundle or a polymer such as Teflon made in a laboratory setting. Regardless, the ligament has to span two points of the native ligament to function.

One of the challenges faced by orthopedic surgeons is how to fix these ligaments securely so that they can function durably and withstand the stresses posed by the patient's activities. The ligament grafts thus have to have extra length at each end so that they can be placed in bone tunnels and secured. The fixation of the graft ends in the bone tunnels can be performed with a variety of techniques including interference fasteners, where a large fastener is used to push the graft up against the bone to facilitate attachment and healing of the graft to the bone. Another technique is to sew the end of the graft to a metal button that is on the outside of the bone. By tying a knot on the button or using a button with an internal cinching mechanism, the sutures coming from the ligament can be kept taut at all times.

The difficulty with the use of buttons is that they require a cortical bridge and are most amenable to a surgical technique where there is only a small aperture in the bone on which the button will rest. The buttons are also problematic in cases of weak cortical bone or in the revision setting where there is a large hole in the cortex of the bone.

### SUMMARY

The various systems and methods of the present disclosure have been developed in response to the present state of the art, and in particular, in response to the problems and needs in the art that have not yet been fully solved by currently available suture fixation systems for ligament reconstruction.

In some embodiments, a suture fixation system for ligament reconstruction configured to tension and secure a ligament graft to a bone using one or more sutures may include a suture lock. The suture lock may include a proximal end including a first portion, a distal end including a second portion that may be smaller than the first portion and configured to be received in a bone tunnel, a ledge portion configured to inhibit the first portion from entering the bone tunnel, and one or more suture apertures extending from the distal end to the proximal end and configured to allow the one or more sutures to pass through the suture lock. The system may further include a fastener including a fastener head and configured to engage the suture lock. With the one or more sutures applying tension to the ligament graft, the fastener may be configured to securably pinch the one or more sutures between the fastener head and the suture lock to inhibit sliding of the one or more sutures relative to the suture lock to maintain the tension of the ligament graft.

In the system of any preceding paragraph, the system may further include a washer including a central aperture configured to receive the fastener and a locking portion. The suture lock may further include a proximal recess, and, with the locking portion engaged in the proximal recess, the washer may be configured to inhibit loosening of the fastener relative to the suture lock.

In the system of any preceding paragraph, the system may further include a suture lock holder having a second distal end, wherein the second distal end may be configured to receive the suture lock and the sutures. The first portion may include a plurality of notches and the second distal end may include a plurality of protrusions configured to be received in the plurality of notches, and the suture lock holder may be configured to removably retain the suture lock and facilitate placement of the suture lock within the bone tunnel.

In the system of any preceding paragraph, the suture lock holder may further include an inside portion configured to allow the one or more sutures to pass through the inside portion with the suture lock retained in the suture lock holder.

In the system of any preceding paragraph, the suture lock may further include a first threaded portion and the fastener may further include a second threaded portion. The second threaded portion may be configured to threadably engage the first threaded portion.

In the system of any preceding paragraph, the second portion may be angularly offset from the first portion so that the second portion aligns with the bone tunnel and the ledge portion aligns with a bone surface proximate the bone tunnel.

In the system of any preceding paragraph, the system may further include a tensioning block including a first surface configured to engage the proximal end of the suture lock and a second surface angularly offset from the first surface. With the tensioning block engaged with the suture lock, a first longitudinal axis of the tensioning block may be parallel with a second longitudinal axis of the second portion.

In some embodiments, a suture fixation system for ligament reconstruction configured to tension and secure a ligament graft to a bone using one or more sutures may include a suture lock. The suture lock may include a proximal end including a first portion, a distal end opposite the proximal end and including a second portion, and one or more suture apertures extending from the distal end to the proximal end and configured to allow the one or more sutures to pass through the suture lock. The system may also include a fastener including a fastener head. The fastener may be configured to engage the suture lock. The system may further include a suture lock holder having a second distal end, wherein the second distal end may be configured to receive the suture lock and the sutures. The first portion may include a plurality of notches, the second distal end may include a plurality of protrusions configured to be received in the plurality of notches, and the suture lock holder may be configured to removably retain the suture lock and facilitate placement of the suture lock within a bone tunnel. With the one or more sutures applying tension to the ligament graft, the fastener may be configured to securably pinch the sutures between the fastener and the suture lock to inhibit sliding of the one or more sutures relative to the suture lock and maintain the tension of the ligament graft

In the system of any preceding paragraph, the system may further include a washer having a central aperture configured to receive the fastener and a locking portion. The washer may be configured to inhibit loosening of the fastener relative to the suture lock and securably pinch the sutures between the locking portion and the suture lock.

In the system of any preceding paragraph, the suture lock holder may further include an inside portion configured to allow the one or more sutures to pass through the inside portion with the suture lock retained in the suture lock holder.

In the system of any preceding paragraph, the second portion may be angularly offset from the first portion so that the second portion aligns with the bone tunnel and the first portion aligns with a bone surface proximate the bone tunnel.

In the system of any preceding paragraph, the system may further include a tensioning block including a first surface configured to engage the proximal end of the suture lock and a second surface angularly offset from the first surface. With the tensioning block engaged with the suture lock, a first longitudinal axis of the tensioning block may be parallel with a second longitudinal axis of the second portion.

In the system of any preceding paragraph, the first surface of the tensioning block may include a plurality of locating bosses. The first portion of the suture lock may further include a plurality of locking apertures, and with the tensioning block engage with the suture lock, the plurality of locating bosses and the plurality of locating apertures may inhibit rotation and translation of the tensioning block relative to the suture lock.

In some embodiments, a suture fixation system for ligament reconstruction configured to tension and secure a ligament graft to a bone using one or more sutures may include a suture lock. The suture lock may include a first portion including a proximal recess and a first longitudinal axis, a second portion that may be smaller than the first portion and may be configured to be received in a bone tunnel and may include a second longitudinal axis that is not parallel to the first longitudinal axis, a ledge portion configured to inhibit the first portion from entering the bone tunnel, and one or more suture apertures extending through the second portion to the proximal recess and configured to allow the one or more sutures to pass through the suture lock. The system may also include a fastener including a fastener head. The fastener may be configured to engage the suture lock. With the one or more sutures applying tension to the ligament graft, the fastener may securably pinch the one or more sutures between the washer and the suture lock to inhibit sliding of the one or more sutures relative to the suture lock and maintain the tension of the ligament graft.

In the system of any preceding paragraph, the suture lock may include an axis angle between the first longitudinal axis and the second longitudinal axis. The axis angle may be between 30 degrees and 70 degrees.

In the system of any preceding paragraph, the washer may include an inside portion configured to receive the fastener and an outside diameter including a locking portion. The locking portion may be configured to securably pinch the one or more sutures between the washer and the suture lock to inhibit the sliding of the one or more sutures relative to the suture lock and maintain the tension of the ligament graft.

In the system of any preceding paragraph, the suture lock may further include a first threaded portion and the fastener may further include a second threaded portion, wherein the second threaded portion may be configured to threadably engage the first threaded portion.

In the system of any preceding paragraph, the system may further include a tensioning block having a first surface configured to engage the first portion of the suture lock and a second surface angularly offset from the first surface. With the tensioning block engaged with the suture lock, a third longitudinal axis of the tensioning block may be parallel with the second longitudinal axis.

In the system of any preceding paragraph, the first surface of the tensioning block may include a plurality of locating bosses, the first portion of the suture lock may further include a plurality of locating apertures, and, with the tensioning block engaged with the suture lock, the plurality of locating bosses and the plurality of locating apertures may inhibit rotation and translation of the tensioning block relative to the suture lock.

In the system of any preceding paragraph, the tensioning block may further include a second central aperture. The fastener head may include a drive feature, and, with the tensioning block engaged with the suture lock, a fastener driver may engage the drive feature through the second central aperture to secure the fastener to the suture block.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings.

FIG. 1A is a perspective view of a suture fixation system according to an embodiment of the present disclosure.

FIG. 1B is an exploded perspective view of the suture fixation system of FIG. 1A.

FIG. 2A is a perspective view of a cortically based suture lock (CBL) according to an embodiment of the present disclosure.

FIG. 2B is a perspective view of the cortically based suture lock (CBL) of FIG. 2A.

FIG. 3A is a perspective view of a spiked obturator tube according to an embodiment of the present disclosure.

FIG. 3B is a perspective view of a dilator tube according to an embodiment of the present disclosure.

FIG. 3C is a perspective view of the spiked obturator tube of FIG. 3A assembled with the dilator tube of FIG. 3B.

FIG. 4A is a perspective view of the spiked obturator tube of FIG. 3A

FIG. 4B is a perspective view of a suture lock holder according to an embodiment of the present disclosure.

FIG. 4C is a perspective view of the spiked obturator tube of FIG. 4A assembled with the suture lock holder of FIG. 4B according to an embodiment of the present disclosure.

FIG. 5A is a perspective view of a locking fastener according to an embodiment of the present disclosure.

FIG. 5B is a perspective view of a washer according to an embodiment of the present disclosure.

FIG. 6 is an exploded perspective view of the suture lock holder of FIG. 4B, the locking fastener of FIG. 5A, the washer of FIG. 5B, and the CBL of FIG. 2A according to an embodiment of the present disclosure.

FIG. 7A is a perspective view of the suture lock holder of FIG. 4B, the locking fastener of FIG. 5A, the washer of FIG. 5B, and the CBL of FIG. 2A in a locked position according to an embodiment of the present disclosure.

FIG. 7B is a perspective view of the suture fixation system of FIG. 1A.

FIG. 8 is a perspective view of the locking fastener of FIG. 5A, the washer of FIG. 5B, a ligament graft and graft sutures being passed through apertures of the CBL of FIG. 2A according to an embodiment of the present disclosure.

FIG. 9A is a perspective view of the suture lock holder of FIG. 4B, the locking fastener of FIG. 5A, the washer of FIG. 5B, a ligament graft and graft sutures being passed through apertures of the CBL of FIG. 2A according to an embodiment of the present disclosure.

FIG. 9B is a perspective view of a fastener driver, the suture lock holder of FIG. 4B, the locking fastener of FIG. 5A, the washer of FIG. 5B, a ligament graft and graft sutures being passed through suture apertures of the CBL of FIG. 2A according to an embodiment of the present disclosure.

FIG. 10A is a perspective view and a detail view of the fastener driver, the suture lock holder of FIG. 4B, the locking fastener of FIG. 5A, the washer of FIG. 5B, a ligament graft and graft sutures being passed through apertures of the CBL of FIG. 2A according to an embodiment of the present disclosure.

FIG. 10B is a perspective view and a detail view of the fastener driver, the suture lock holder of FIG. 4B, the locking fastener of FIG. 5A, the washer of FIG. 5B, a ligament graft and graft sutures being passed through apertures of the CBL of FIG. 2A according to an embodiment of the present disclosure.

FIG. 11 is a perspective view and a detail view of a bone, the locking fastener of FIG. 5A, the washer of FIG. 5B, a ligament graft and graft sutures being passed through apertures of the CBL of FIG. 2A, with the CBL of FIG. 2A in a locked position, according to an embodiment of the present disclosure.

FIG. 12A is a perspective view of a revision cortically based suture lock (RCBL) according to an embodiment of the present disclosure.

FIG. 12B is a perspective view of the RCBL of FIG. 12A.

FIG. 12C is a perspective view of the RCBL of FIG. 12A.

FIG. 13A is a perspective view of a tensioning block according to an embodiment of the present disclosure.

FIG. 13B is a perspective view of the tensioning block of FIG. 13A.

FIG. 14A is a perspective view of the RCBL of FIG. 12A and the tensioning block of FIG. 13A prior to engagement according to an embodiment of the present disclosure.

FIG. 14B is a perspective view of the RCBL of FIG. 12A and the tensioning block of FIG. 13A prior to engagement according to an embodiment of the present disclosure.

FIG. 15 is a perspective view of the RCBL of FIG. 12A and the tensioning block of FIG. 13A after engagement of the two components according to an embodiment of the present disclosure.

FIG. 16 is a perspective view of a bone tunnel prepared for placement of the RCBL of FIG. 12A according to an embodiment of the present disclosure.

FIG. 17 is a perspective view of the bone tunnel of FIG. 16 after placement of the RCBL of FIG. 12A and prior to final placement of the washer of FIG. 5B and the locking fastener of FIG. 5A according to an embodiment of the present disclosure.

FIG. 18 is a perspective view of the bone tunnel of FIG. 16 after placement of the RCBL of FIG. 12A and prior to final locking of the washer of FIG. 5B and the locking fastener of FIG. 5A and with passage of sutures through the suture apertures of the RCBL of FIG. 12A according to an embodiment of the present disclosure.

FIG. 19 is a perspective view of the bone tunnel of FIG. 16 after placement of the RCBL of FIG. 12A and the tensioning block of FIG. 13A with sutures tied around a tensioning tool according to an embodiment of the present disclosure.

FIG. 20 is a perspective view of the fastener driver of FIG. 9B, the bone tunnel of FIG. 16 after placement of the RCBL of FIG. 12A and the tensioning block of FIG. 13A with sutures tied around the tensioning tool of FIG. 19 according to an embodiment of the present disclosure.

FIG. 21 is a perspective view of the bone tunnel of FIG. 16 after placement of the RCBL of FIG. 12A and after tensioning of the sutures and final tightening of the locking fastener of FIG. 5A and after removal of the fastener driver of FIG. 9B according to an embodiment of the present disclosure.

FIG. 22 is an exemplary method for ligament reconstruction using a suture fixation system according to an embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to suture fixation systems for ligament reconstruction. Those skilled in the art will recognize that the following description is merely illustrative of the principles of the technology, which may be applied in various ways to provide many alternative embodiments. For the purposes of illustrating the concepts of the present design, the present disclosure describes suture fixation systems for ACL reconstruction. However, it will be understood that other variations and uses are contemplated, including, but not limited to, applications in PCL reconstruction, lateral ankle ligament reconstruction, carpometacarpal (CMC) ligament reconstruction, hip ligamentum teres reconstruction, and/or reconstruction of other ligaments associated with a joint.

The present disclosure relates to a device and technique for repair of ligaments using grafts with sutures holding each end of the graft. A system according to the present disclosure may include a method and a device for stabilizing a suture, in an adjustable fashion, to a fixation device to allow stable fixation and appropriate tension of a ligament.

A system according to the present disclosure may include a cortically based suture lock (CBL) placed at least partially within and/or proximate a bone tunnel. A fastener may be disposed within a central aperture of the CBL and may function as a locking fastener. A fastener channel may be surrounded by one to several apertures for passage of sutures. A locking fastener may include a tapered or flat head such that, by tightening the locking fastener into the central aperture, it may lock the sutures in place at whatever position and tension they are held. This mechanism may also be augmented through the use of a washer.

The washer may be constructed of a metal or a polymer in order to minimize damage to the sutures upon locking. The device may be placed at least partially within and/or proximate a bone tunnel such as one produced in a tibia and/or femur for ACL reconstruction. Additionally, or alternatively, the device may be placed within a bone tunnel as part of a procedure for reconstruction of other ligaments, such as, a posterior cruciate ligament (PCL), a lateral ankle ligament, a carpometacarpal (CMC) ligament, hip ligamentum teres reconstruction, and/or other ligaments associated with a joint.

The sutures may be secured to the ligament graft and passed through apertures in the CBL prior to placement of the locking fastener into the CBL. With the fastener placed in the CBL, the sutures may be tensioned. Tensioning of the sutures may be performed with a suture tensioning tool. With the sutures under tension, the locking fastener and a washer may be used to compress the sutures against an internal wall of the CBL. The sutures may be tensioned by hand or through the use of a tensioning device.

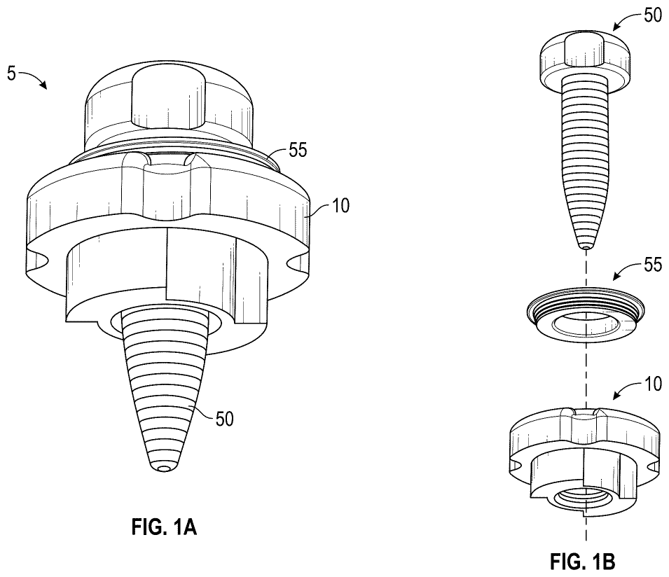

FIG. 1A is a perspective view of a suture fixation system 5 according to an embodiment of the present disclosure. FIG. 1B is an exploded perspective view of the suture fixation system 5. The suture fixation system 5 may be configured to facilitate fixation of a ligament to a bone. The suture fixation system 5 may securably receive one or more sutures and/or suture ends. The one or more sutures and/or suture ends may extend from a ligament graft and may be secured under tension to the suture fixation system 5.

The suture fixation system 5 may be configured to ensure desired tension of an autogenous, a cadaveric (allograft), and/or a synthetic ligament graft via one or more sutures and/or suture tapes linking the suture fixation system 5 to the ligament graft. The one or more sutures and/or suture tapes may be secured to the ligament graft at a first suture end and/or suture tape end and may be secured to the suture fixation system 5 at or near a second suture end and/or suture tape end opposite the first suture end and/or the first suture tape end. The suture fixation system 5 may be configured to prevent a cortically based suture lock (CBL) 10 from migrating inwards from a bone cortex towards an interior of the bone as a result of tension in the suture and/or suture tape that is secured to the suture fixation system 5.

The suture fixation system 5 may include the CBL 10, a locking fastener 50, and a washer 55. The CBL 10 may be configured to receive the locking fastener 50 and the washer 55. The washer 55 may be configured to receive the locking fastener 50. The suture fixation system 5 may be configured to facilitate fixation of a ligament to a bone.

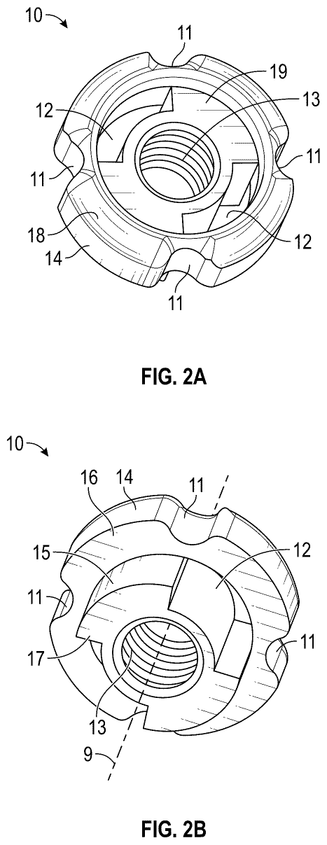

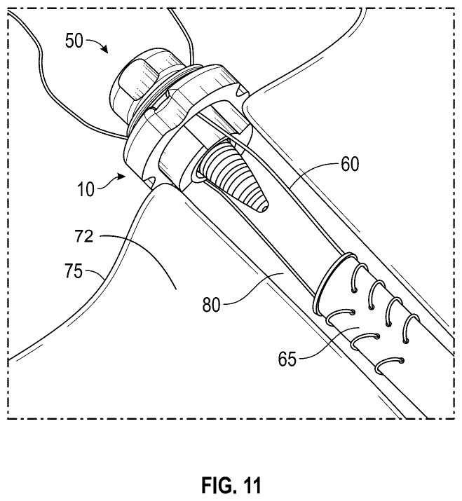

FIG. 2A is a perspective view of a CBL 10 according to an embodiment of the present disclosure. FIG. 2B is a perspective view of the CBL 10. The CBL 10 may be configured to be received within a bone tunnel 80 proximate a bone cortex 75, as shown in FIG. 11. The CBL 10 may be further configured so that the CBL 10 remains proximate the bone cortex 75 when a graft suture 60 is secured to the suture fixation system 5 and tension is applied to the graft suture 60 (also shown in FIG. 11).

The CBL 10 may include a central axis 9, one or more suture apertures 12, an internal thread 13, a first outside portion 14, a second outside portion 15, and a ledge portion 16. The apertures 12 may extend from a proximal end 18 to a distal end 17. The apertures 12 may be configured to slidably receive one or more sutures and/or a suture tapes. The apertures 12 may allow the one or more sutures and/or suture tapes to pass through the CBL 10 from the distal end 17 to the proximal end 18. Alternatively, the apertures 12 may allow the one or more sutures and/or suture tapes to pass through the CBL 10 from the proximal end 18 to the distal end 17.

Each of the apertures 12 may have an arcuate shape and may be positioned between the first outside portion 14 and the internal thread 13. The arcuate shape may be generally concentric with the first outside portion 14. Each of the apertures 12 may be sized to receive multiple sutures and/or suture tapes. The size of the apertures 12 may facilitate easy insertion and flexibility of the sutures and/or suture tapes.

The apertures 12 may be distributed equally about the central axis 9. In an embodiment, the CBL 10 may include two, four, or more suture apertures 12. More specifically, the CBL 10 may include two suture apertures 12 spaced approximately 180 degrees apart. The apertures 12 may be sized to receive a suture from size 4-0 through size 5. Additionally, or alternatively, the apertures 12 may be sized to receive a suture tape having a width from 0.8 mm through 5 mm.

The internal thread 13 may extend from the proximal end 18 to the distal end 17 and may be configured to threadably receive the locking fastener 50. The internal thread 13 may be generally concentric to the central axis 9.

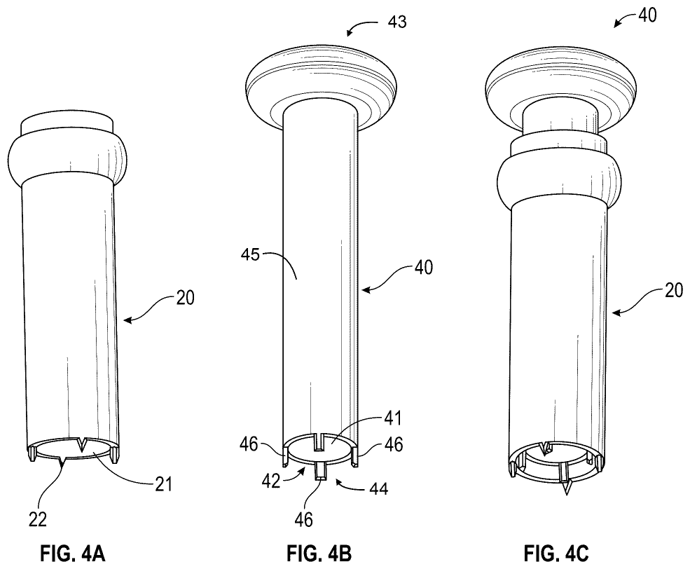

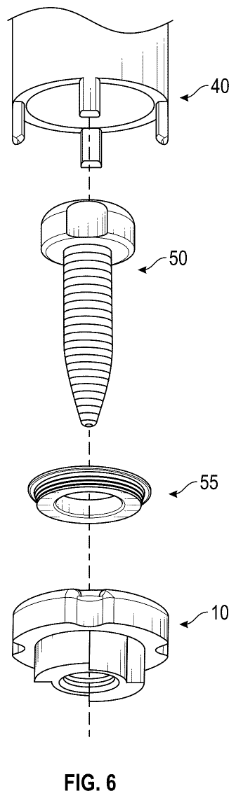

The first outside portion 14 may include a plurality of notches 11. The plurality of notches 11 may be configured to engage a suture lock holder 40 (shown in FIGS. 4B, 4C, and 6) to allow rotational control of the CBL 10. The first outside portion 14 may be generally circular with the plurality of notches 11 equally spaced around the circumference. Alternatively, the first outside portion 14 may include a non-circular shape, such as a square or hexagon. The CBL 10 may include between two and ten notches 11. In an embodiment, the CBL 10 may include four notches 11 spaced generally 90 degrees apart.

The second outside portion 15 may be sized to be received within a bone tunnel. The second outside portion 15 may be smaller than the first outside portion 14, thereby creating the ledge portion 16. The ledge portion 16 may be configured to abut a bone cortex when the second outside portion 15 is received within the bone tunnel. The ledge portion 16 may inhibit the first outside portion 14 from entering the bone tunnel.

The proximal end 18 may include a proximal recess 19. The proximal recess 19 may be configured to receive the washer 55. Additionally, or alternatively, the proximal recess 19 may be configured to receive a fastener head 53 of the locking fastener 50.

The CBL 10 may be configured such that the second outside portion 15 is sized to have a diameter within a range of 4 mm to 20 mm. More specifically, the second outside portion 15 may be sized to have a diameter within a range of 7 mm to 15 mm. More specifically, the second outside portion 15 may be sized to have a diameter within a range of 9 mm to 13 mm. More specifically, the second outside portion 15 may be sized to have a diameter of 11 mm.

The CBL 10 may be configured such that the first outside portion 14 may be sized to have a diameter within a range of 7 mm to 25 mm. More specifically, the first outside portion 14 may be sized to have a diameter within a range of 12 mm to 20 mm. More specifically, the first outside portion 14 may be sized to have a diameter of 16 mm. More specifically, the first outside portion 14 may be sized to have a diameter within a range of 3 mm to 7 mm larger than the second outside portion 15. More specifically, the first outside portion 14 may be sized to have a diameter 5 mm larger than the second outside portion 15. In an embodiment, the CBL 10 may be one of a set of differently-sized cortically based suture locks (CBL), each having a differently sized first outside portion 14 and/or second outside portion 15.

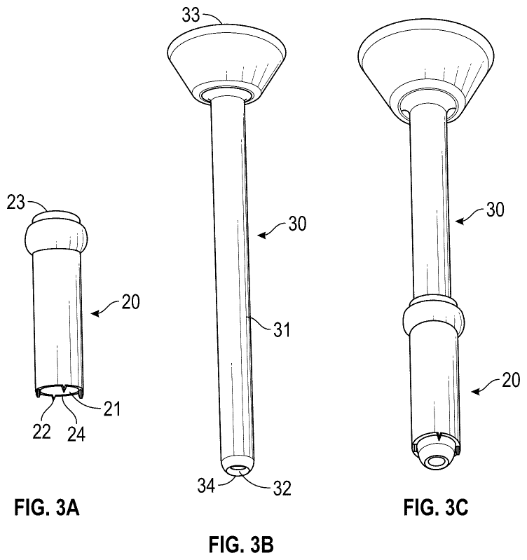

FIG. 3A is a perspective view of a spiked obturator 20 according to an embodiment of the present disclosure. The spiked obturator 20 may allow for unobstructed entry of the CBL 10 and may act as a soft tissue retractor. The spiked obturator 20 may be configured to receive a dilator tube 30. Additionally, the spiked obturator 20 may be configured to receive a suture lock holder 40 and a CBL 10.

The spiked obturator 20 may include a proximal end 23, a distal end 24, and a bore that defines an inside diameter 21 extending from the proximal end 23 to the distal end 24. The inside diameter 21 may be sized to receive the dilator tube 30, the suture lock holder 40, and/or the cortically based suture lock (CBL) 10. The distal end 24 may include one or more spikes 22. The one or more spikes 22 may be configured engage a surface of a bone to inhibit movement of the spiked obturator 20 relative of the surface of the bone.

FIG. 3B is a perspective view of a dilator tube 30 according to an embodiment of the present disclosure. FIG. 3C is a perspective view of the spiked obturator 20 assembled with the dilator tube 30. The dilator tube 30 may be configured to dilated soft tissue and facilitate placement of the spiked obturator 20 on the surface of the bone at a planned placement site of the CBL 10.

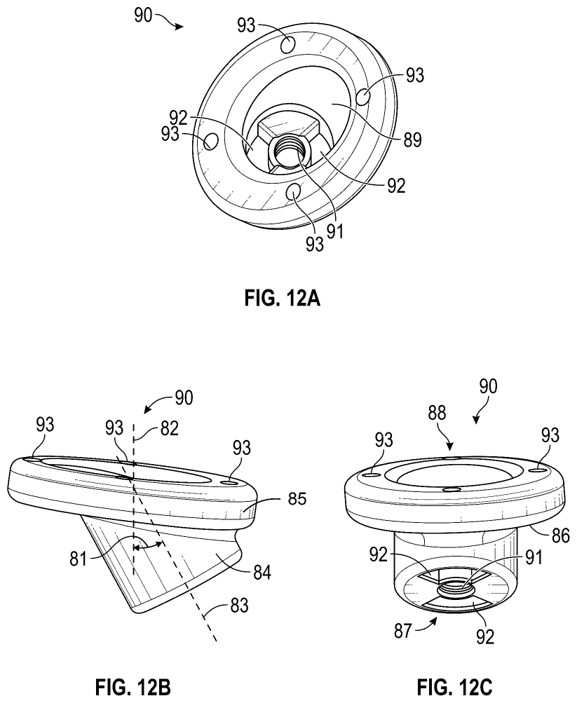

The dilator tube 30 may include a proximal end 33, a distal end 34, a shaft portion 31 between the proximal end 33 and the distal end 34, and a bore defining an inside diameter 32 extending from the proximal end 33 to the distal end 34. The shaft portion 31 may be sized to be slidably received within the inside diameter 21 of the spiked obturator 20. The inside diameter 32 may be configured to receive a guidewire (not shown) having a diameter between 3 mm and 5 mm. The guidewire may be inserted through the proximal skin, through the bone, and into the joint at the desired attachment point of the reconstructed ligament to the bone, thereby identifying a trajectory of the desired bone tunnel. The trajectory may be confirmed using fluoroscopy, arthroscopic examination, and/or direct visualization prior to dilating the soft tissue and/or creating the bone tunnel. By passing the guidewire through bone and into the joint, a location for each end of the bone tunnel may be identified. In a case in which the bone tunnel is not generally perpendicular to the surface of the bone, a reamer may be used to create a counterbore, that is perpendicular to the bone tunnel, in which the CBL may be placed so that a longitudinal axis of the CBL may be parallel to a longitudinal axis of the bone tunnel. Alternatively, in a case in which the bone tunnel is not generally perpendicular to the surface of the bone, or a previous larger bone tunnel is present as in the case of a revision procedure, a revision cortically based suture lock (RCBL) may be used as shown in FIGS. 12A through 21.

With the dilator tube 30 received within the spiked obturator 20, the dilator tube 30 and the spiked obturator 20 may be passed over the guidewire to the planned placement site of the CBL 10. With the one or more spikes 22 engaged with the surface of the bone, the dilator tube 30 and the guidewire may be withdrawn, leaving the spiked obturator in place to function as a cannula through which other instruments and/or implant may be guided to the planned placement site of the CBL 10.

FIG. 4A is a perspective view of the spiked obturator 20. FIG. 4B is a perspective view of a suture lock holder 40 according to an embodiment of the present disclosure. FIG. 4C is a perspective view of the spiked obturator 20 assembled with the suture lock holder 40 according to an embodiment of the present disclosure. The suture lock holder 40 may be configured to removably retain the CBL 10 and may facilitate placement of the CBL 10 within a bone tunnel. The suture lock holder 40 may further be configured to act as a counter-torque to inhibit rotation of the CBL 10 as the locking fastener 50 threadably engages the CBL 10.

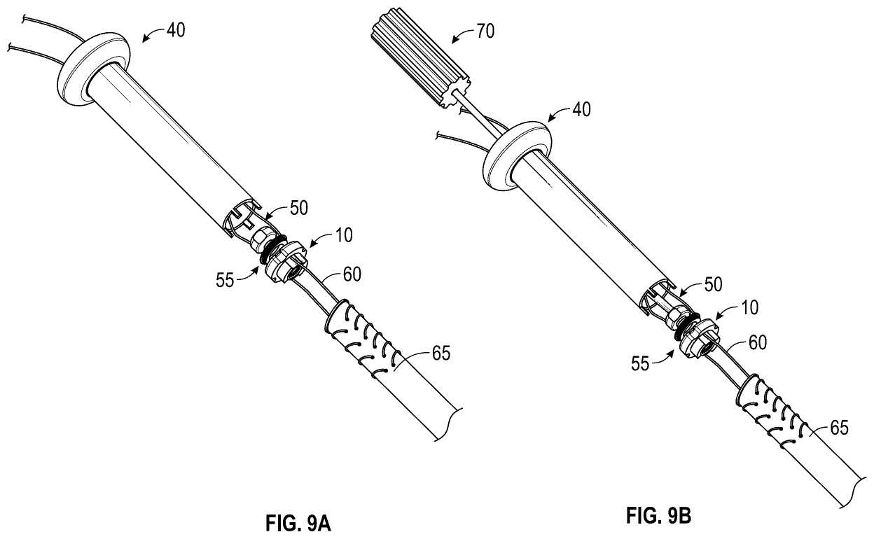

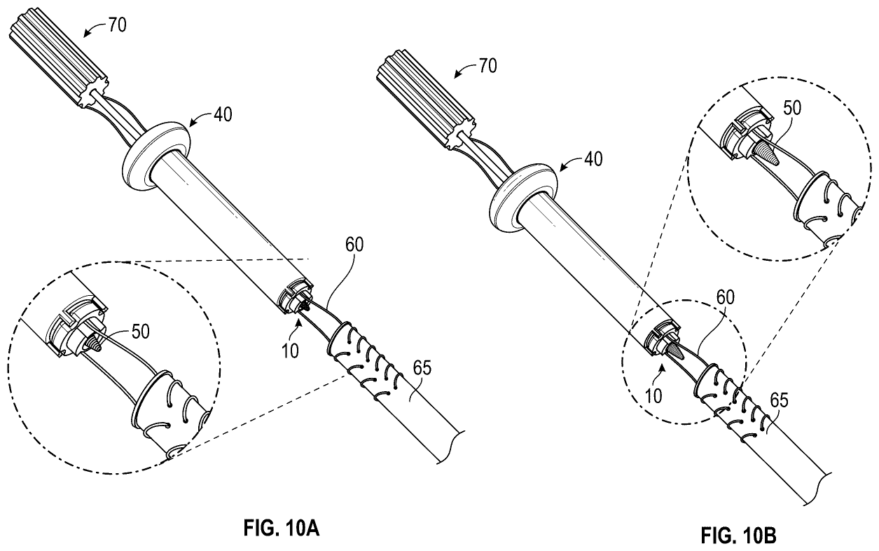

The suture lock holder 40 may include a proximal end 43, a distal end 44, a bore defining an inside portion 41 extending from the proximal end 43 to the distal end 44, and a shaft portion 45 between the proximal end 43 and the distal end 44. The shaft portion 45 may be sized to be slidably received within the inside diameter 21 of the spiked obturator 20. The inside portion 41 may be configured to receive the locking fastener 50, the washer 55, sutures, and/or the distal end of a fastener driver 70 (shown in FIGS. 9B, 10A, and 10B).

The distal end 44 may include a drive feature 42. The drive feature 42 may include a plurality of protrusions 46. The drive feature 42 may be configured to engage the plurality of notches 11 of the CBL 10 to allow rotational control of the CBL 10. The drive feature 42 may include between 2 and ten protrusions 46. The number of protrusions may be equal to or less than the number of notches 11. The plurality of protrusions 46 may be arranged to allow engagement of the drive feature 42 with the CBL 10. In an embodiment, the drive feature 42 may include four protrusions 46 spaced generally 90 degrees apart.

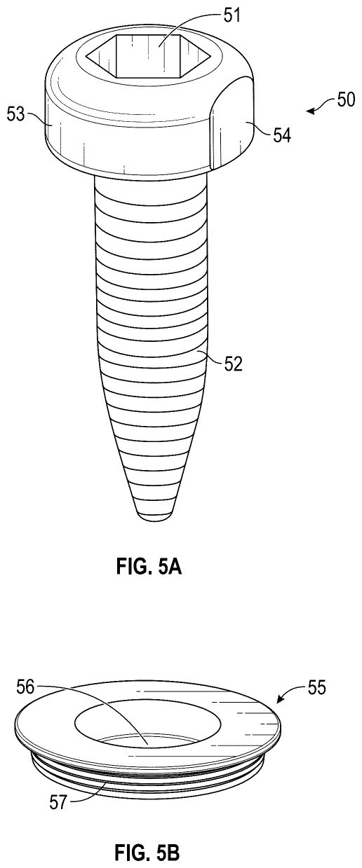

FIG. 5A is a perspective view of a locking fastener 50 according to an embodiment of the present disclosure. The locking fastener 50 may be configured to secure one or more sutures, and/or one or more suture tapes to the CBL 10. In an embodiment, the locking fastener 50 may be configured as a screw. Alternatively, the locking fastener 50 may be configured as a brad, a Christmas type fastener, a j-lock, a cam lock, and/or other fastener type known in the art. The locking fastener 50 is shown as an internal fastener, however, it is also contemplated that the locking fastener may be configured as an external fastener, for example, the fastener may be configured as a nut and the CBL may include external threads.

The locking fastener 50 may include a drive feature 51, a threaded portion 52, and a fastener head 53. The drive feature 51 may be included in the fastener head 53. The drive feature 51 may facilitate rotation of the locking fastener 50 through engagement with the fastener driver 70. The drive feature 51 may be configured as a hex, a hexalobe, a square or other non-circular profile known in the art. The threaded portion 52 may correspond to the internal thread 13 of the CBL 10 so that the locking fastener 50 may threadably engage the CBL 10.

The fastener head 53 may be sized to have a larger diameter than the threaded portion 52. The fastener head 53 may be generally circular and may include one or more flats 54 that facilitate insertion and/or rotation of the fastener 50 into the CBL 10. Alternatively, the fastener head 53 may have a non-circular geometry, such as a square, a hexagon, or other non-circular geometry. The fastener head 53 may be configured to apply pressure between the fastener head 53 and the washer 55 and/or the proximal recess 19 of the cortically based suture lock (CBL) 10. In response to threadably tightening the locking fastener 50 relative to the CBL 10, the fastener head 53 may secure one or more sutures, and/or one or more suture tapes to prevent movement of the one or more sutures, and/or one or more suture tapes relative to the suture fixation system 5.

The locking fastener 50 may be configured to securably pinch one or more sutures, and/or one or more suture tapes between the fastener head 53 and the washer 55. Additionally, or alternatively, the locking fastener 50 may be configured to securably pinch one or more sutures, and/or one or more suture tapes between the fastener head 53 and the CBL 10.

FIG. 5B is a perspective view of a washer 55 according to an embodiment of the present disclosure. The washer 55 may be configured to inhibit loosening of the locking fastener 50 relative to the CBL 10 after threadably tightening the locking fastener 50 within the CBL 10. Additionally, or alternatively, in response to threadably tightening the locking fastener 50 relative to the CBL 10, the fastener head 53 may secure one or more sutures, and/or one or more suture tapes securely against the washer 55. Additionally, or alternatively, in response to threadably tightening the locking fastener 50 relative to the CBL 10, the fastener head 53 may apply a force to the washer 55 and, as a result, the washer 55 may secure one or more sutures, and/or one or more suture tapes securely against the CBL 10.

The washer 55 may include a central aperture 56 and a locking portion 57. The central aperture 56 may be sized to receive the threaded portion 52 of the locking fastener 50. The central aperture 56 may be further configured so that one or more sutures, and/or one or more suture tapes that may pass through the apertures 12 may also pass through the central aperture 56 with the washer 55 received within the proximal recess 19. The central aperture 56 may have an inside diameter that may be less than an outside diameter of the fastener head 53.

The locking portion 57 may include a circumferential groove, a radial groove, serrations, and/or a surface texture to inhibit movement of the washer 55 relative to the CBL 10 and/or inhibit motion of the suture or suture tape relative to the washer 55 after final tightening of the locking fastener 50 in the CBL 10. Additionally, or alternatively, the locking portion 57 may be received within the proximal recess 19 and may be configured to securely pinch one or more sutures, and/or one or more suture tapes between the locking portion 57 and the proximal recess 19 in response to final tightening of the locking fastener 50. Additionally, or alternatively, the proximal recess 19 may include a locking portion that may be configured to interdigitate with the locking portion 57. In an embodiment, the proximal recess 19 may include a locking portion, similar to the locking portion 57, and the washer 55 may not include a locking portion, thereby securely pinching one or more sutures, and/or one or more suture tapes between the locking portion of the proximal recess 19 and the washer 55 in response to final tightening of the locking fastener 50.

The locking portion 57 may be configured to securely pinch the one or more sutures, and/or one or more suture tapes between the locking portion 57 and the proximal recess 19 in an atraumatic manner to limit damage to the one or more sutures, one or more suture ends, one or more suture tapes, and/or one or more suture tape ends.

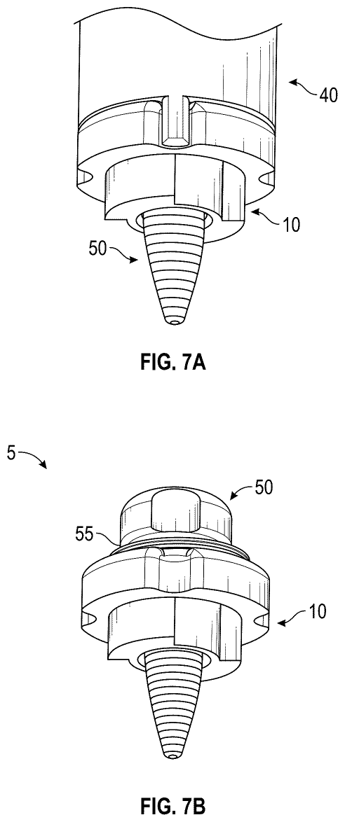

FIG. 6 is an exploded perspective view of the suture lock holder 40, the locking fastener 50, the washer 55, and the CBL 10 according to an embodiment of the present disclosure. FIG. 7A is a perspective view of the suture lock holder 40, the locking fastener 50, the washer 55, and the CBL 10 in a locked position according to an embodiment of the present disclosure. FIG. 7B is a perspective view of the suture fixation system 5.

The drive feature 42 of the suture lock holder 40 may be received with the notches 11 of the CBL 10, thereby engaging the CBL 10 within the suture lock holder 40. With the CBL 10 engaged within the suture lock holder 40, the locking fastener 50 and the washer 55 may be received within the inside portion 41 of the suture lock holder 40. The suture lock holder 40 may be used to insert the CBL 10 into the bone tunnel. Additionally, the suture lock holder 40 may be used to adjust, orient, and/or rotate the CBL 10 relative to the bone tunnel.

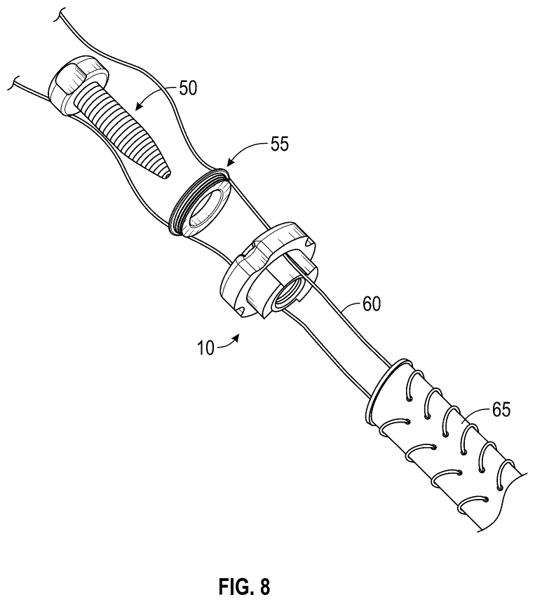

FIG. 8 is a perspective view of the locking fastener 50, the washer 55, a ligament graft 65 and graft sutures 60 being passed through apertures of the CBL 10 according to an embodiment of the present disclosure. The graft sutures 60 may be secured to a ligament graft 65 using any suturing technique known in the art, such as Krackow, McKeon's Double Krackow, Wilson's Double Krackow, Ostrander's Modified Krackow, Giftbox-modified Krackow, Kessler, or Bunnell. The ends of the graft sutures 60 may then be passed through the apertures 12 of the CBL 10. The ends of the graft sutures 60 may be passed through the apertures 12 prior to engagement of the locking fastener 50 and/or the washer 55 with the CBL 10.

FIG. 9A is a perspective view of the suture lock holder 40, the locking fastener 50, the washer 55, a ligament graft 65 and graft sutures 60 being passed through the apertures 12 of the CBL 10 according to an embodiment of the present disclosure. FIG. 9B is a perspective view of a fastener driver 70, the suture lock holder 40, the locking fastener 50, the washer 55, a ligament graft 65 and graft sutures 60 being passed through the apertures 12 of the CBL 10 according to an embodiment of the present disclosure.

With the ends of the graft sutures 60 may be passed through the apertures 12, the locking fastener 50 may be passed through the washer 55 and threadably engage the CBL 10, note: the locking fastener 50 may not be fully tightened at this point. The ends of the graft sutures 60 may also be passed through the inside portion 41 of the suture lock holder 40. In this state, the suture fixation system 5 may slide along the ends of the graft sutures 60 without damaging the ends of the graft sutures 60. The fastener driver 70 may be introduced through the inside portion 41 of the suture lock holder 40 to engage the drive feature 51 of the locking fastener 50.

FIG. 10A is a perspective view and a detail view of the fastener driver 70, the Suture lock holder 40, the locking fastener 50, the washer 55, a ligament graft 65 and graft sutures 60 being passed through apertures of the CBL 10 according to an embodiment of the present disclosure. The suture lock holder 40 may engage the suture fixation system 5 and may guide the CBL 10 into the bone tunnel. The suture fixation system 5 may slide along the ends of the graft sutures 60 to facilitate tensioning of the ligament graft 65. The CBL may be placed on the bone prior to tensioning the sutures 60 and/or tightening the locking fastener 50. The bone is not shown in FIGS. 10A and 10B for clarity.

FIG. 10B is a perspective view and a detail view of the fastener driver 70, the suture lock holder 40, the locking fastener 50, the washer 55, a ligament graft 65 and graft sutures 60 being passed through apertures of the CBL 10 according to an embodiment of the present disclosure. With the ligament graft 65 tensioned the desired amount, the fastener driver 70 may engage the locking fastener 50 and may threadably tighten the locking fastener 50 into the CBL 10 so that the graft sutures 60 may be secured to the suture fixation system 5, precluding any sliding of the graft sutures 60 relative to the suture fixation system 5, thereby locking the tension of the ligament graft 65.

FIG. 11 is a perspective view and a detail view of a bone 72, the locking fastener 50, the washer 55, a ligament graft 65 and graft sutures 60 being passed through apertures of the CBL 10, with the CBL 10 in a locked position, according to an embodiment of the present disclosure. The bone 72 may include a bone cortex 75 and a bone tunnel 80. The CBL 10 may be inserted into the bone tunnel 80 so that the ledge portion 16 may abut the bone cortex 75. The tensioned ligament graft 65 and the graft sutures 60 may exert a force on the suture fixation system 5 toward the ligament graft 65, however, the ledge portion 16 and the bone cortex 75 may inhibit the suture fixation system 5 from moving towards the ligament graft 65.

In some instances, a reconstructed ligament graft may fail due to a reinjury of the ligament, problems arising from a previous procedure, failure of the reconstructed ligament to heal properly, and/or other factors that inhibit the ligament from functioning properly. In these cases, a revision ligament graft reconstruction procedure may be necessary. Revision graft reconstruction may necessitate creating a bone tunnel that receives the suture fixation system, in a location that is different from a bone tunnel used for a previous ligament graft reconstruction procedure. For revision ligament graft reconstruction surgery, it may be necessary to create the revision bone tunnel at an acute angle relative to a surface of the bone. During revision ligament graft reconstruction surgery, a previous ligament graft and/or implant may be removed and/or a previous bone tunnel may require bone grafting.

FIG. 12A is a perspective view of a revision cortically based suture lock (RCBL) 90 according to an embodiment of the present disclosure. FIG. 12B is a perspective view of the RCBL 90 and FIG. 12C is a perspective view of the RCBL 90. A revision suture fixation system 6 may include the RCBL 90, and a locking fastener and a washer, which may optionally be similar or identical to the locking fastener 50 and the washer 55.

The revision suture fixation system 6 may be configured to facilitate fixation of a ligament to a bone. The revision suture fixation system 6 may further be configured for revision ligament graft reconstruction surgery, such as revision ACL reconstruction, revision PCL reconstruction, revision lateral ankle ligament reconstruction, and/or revision reconstruction of other ligaments associated with a joint. The revision suture fixation system 6 may securably receive one or more sutures. The one or more sutures and/or suture ends may extend from a ligament graft and may be secured under tension to the revision suture fixation system 6.

The revision suture fixation system 6 may be configured to ensure desired tension of an autogenous, a cadaveric, autograft, and/or a synthetic ligament graft via one or more sutures and/or suture tapes linking the revision suture fixation system 6 to the ligament graft. The one or more sutures and/or suture tapes may be secured to the ligament graft at a first suture end and/or suture tape end and may be secured to the revision suture fixation system 6 at a second suture end and/or suture tape end opposite the first suture end and/or suture tape end. The revision suture fixation system 6 may be configured to prevent a revision cortically based suture lock (RCBL) 90 from migrating inwards from a bone cortex towards an interior of the bone as a result of tension in the suture and/or suture tape that is secured to the revision suture fixation system 6.

The RCBL 90 may be configured to receive the locking fastener 50 and the washer 55. The washer 55 may be configured to receive the locking fastener 50. The revision suture fixation system 6 may be configured to facilitate fixation of a ligament to a bone. The RCBL 90 may be configured to be received within a bone tunnel 80 proximate a bone cortex 75. The RCBL 90 may be further configured so that the RCBL 90 remains proximate the bone cortex 75 when a graft suture 60 is secured to the revision suture fixation system 6 and tension is applied to the graft suture 60.

The RCBL 90 may include one or more suture apertures 92, an internal thread 91, a first outside portion 85, a second outside portion 84, and a ledge portion 86. The first outside portion 85 may be centered about a first axis 82. The second outside portion 84 may be centered about a second axis 83. The first axis 82 and the second axis are offset from each other at an axis angle 81. The second outside portion 84 may be angularly offset from the first outside portion 85 so that the second outside portion 84 aligns with the bone tunnel and the ledge portion 86 aligns with a bone surface proximate the bone tunnel.

The first outside portion 85 may be sized to have a diameter within a range of 7 mm to 25 mm. More specifically, the first outside portion 85 may be sized to have a diameter within a range of 12 mm to 20 mm. More specifically, the first outside portion 85 may be sized to have a diameter of 16 mm.

The second outside portion 84 may be sized to have a diameter within a range of 4 mm to 20 mm. More specifically, the second outside portion 84 may be sized to have a diameter within a range of 7 mm to 15 mm. More specifically, the second outside portion 84 may be sized to have a diameter within a range of 9 mm to 13 mm. More specifically, the second outside portion 84 may be sized to have a diameter of 11 mm. More specifically, the first outside portion 85 may be sized to have a diameter within a range of 3 mm to 7 mm larger than the second outside portion 84. More specifically, the first outside portion 85 may be sized to have a diameter 5 mm larger than the second outside portion 84.

The RCBL 90 may be configured such that the axis angle 81 may be within a range of 0 degrees to 80 degrees. More specifically, the axis angle 81 may be within a range of 30 degrees to 70 degrees More specifically, the axis angle 81 may be within a range of 40 degrees to 60 degrees. In an embodiment, the RCBL 90 may be one of a set of differently-sized revision cortically based suture locks (RCBL), each having a different axis angle 81, differently sized first outside portion 85, and/or a differently sized second outside portion 84. The RCBL may be chosen so that the axis angle 81 is generally equal to an angle between a vector normal to the bone surface and an axis of the bone tunnel.

The one or more suture apertures 92 may extend from a distal end 87 through a proximal end 88. The apertures 92 may be configured to receive one or more sutures and/or a suture tapes. The apertures 92 may allow the one or more sutures and/or suture tapes to pass through the RCBL 90 from the distal end 87 through the proximal end 88. Alternatively, the apertures 92 may allow the one or more sutures and/or suture tapes to pass through the RCBL 90 from the distal end 87 through the proximal end 88.

The apertures 92 may be distributed equally about the first axis 82. In an embodiment, the RCBL 90 may include between two and four suture apertures 92. More specifically, the RCBL 90 may include two suture apertures 92 spaced approximately 180 degrees apart. The apertures 92 may be sized to receive a suture from size 4-0 through size 5. Additionally, or alternatively, the apertures 92 may be sized to receive a suture tape having a width from 0.8 mm through 5 mm.

The internal thread 91 may extend from the distal end 87 through the proximal end 88 and may be configured to threadably receive the locking fastener 50. The internal thread 91 may be generally concentric to the second axis 83.

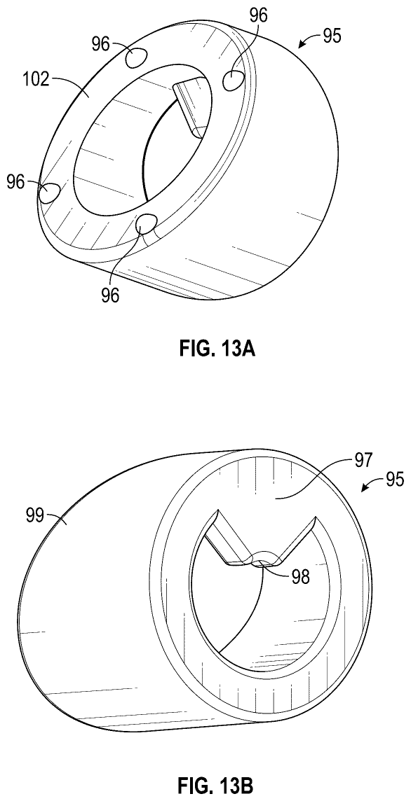

The first outside portion 85 may include a plurality of locating apertures 93. The plurality of locating apertures 93 may be configured to engage a tensioning block 95 (shown in FIGS. 13A-15) to inhibit rotation and translation of the RCBL 90 relative to the tensioning block 95. The first outside portion 85 may be generally circular. Alternatively, the first outside portion 85 may include a non-circular shape, such as a square or hexagon. The RCBL 90 may include between two and ten locating apertures 93. In an embodiment, the RCBL 90 may include four locating apertures 93 spaced generally 90 degrees apart.

The second outside portion 84 may be sized to be received within a bone tunnel. The second outside portion 84 may be smaller than the first outside portion 85, thereby creating the ledge portion 86. The ledge portion 86 may be configured to abut a bone cortex when the second outside portion 84 is received within the bone tunnel. The ledge portion 86 may inhibit the first outside portion 85 from entering the bone tunnel.

The proximal end 88 may include a proximal recess 89. The proximal recess 89 may be configured to receive the washer 55. Additionally, or alternatively, the proximal recess 89 may be configured to receive the fastener head 53 of the locking fastener 50.

FIG. 13A is a perspective view of a tensioning block 95 according to an embodiment of the present disclosure. FIG. 13B is a perspective view of the tensioning block 95. The tensioning block 95 may be configured to engage the RCBL 90 during engagement of the locking fastener 50 with the RCBL 90 and/or tensioning of the one or more sutures and/or suture tapes.

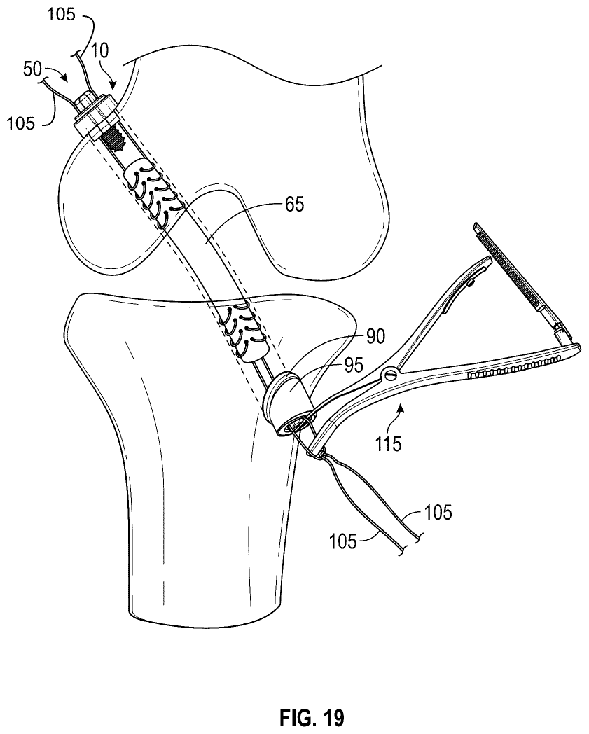

The tensioning block 95 may provide a surface that is perpendicular to a longitudinal axis of the bone tunnel so that a tensioning tool (shown in FIG. 19) may act on the surface to apply tension to the sutures. The CBL does not have an axis angle between the first outside portion and the second outside portion and, therefore, a proximal surface of the CBL is perpendicular with a longitudinal axis of the bone tunnel, therefore, the tensioning block may not be needed to tension sutures when implanting the CBL. The tensioning block 95 may be removed after final tightening of the locking fastener 50 and may not be implanted as part of the revision suture fixation system 6.

The tensioning block 95 may include a plurality of locating bosses 96, a proximal surface 97, a central aperture 98, and a central axis 103. The plurality of locating bosses 96 may be located on a distal surface 102 opposite the proximal surface 97. The proximal surface 97 may not be parallel to the distal surface 102. The central aperture 98 may extend from the proximal surface 97 through the distal surface 102. The plurality of locating bosses 96 may be configured to engage the plurality of locating apertures 93 to inhibit rotation of the RCBL 90 and sliding of the tensioning block 95 relative to the RCBL 90 during suture tensioning. The tensioning block 95 may include between two and ten locating bosses 96. In an embodiment, the tensioning block 95 may include four locating bosses 96 spaced generally 90 degrees apart.

The tensioning block 95 may also include an outside portion 99 that may be generally sized and shaped to match the first outside portion 85 of the RCBL 90. The central aperture 98 may be configured so that the one or more sutures and/or suture tapes may pass through. Additionally, the central aperture 98 may be configured so that, with the tensioning block 95 and the locking fastener 50 engaged with the RCBL 90, the fastener driver 70 may engage the locking fastener 50 to threadably engage the locking fastener 50 with the RCBL 90.

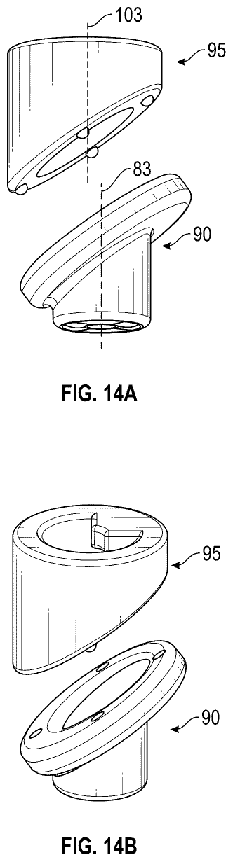

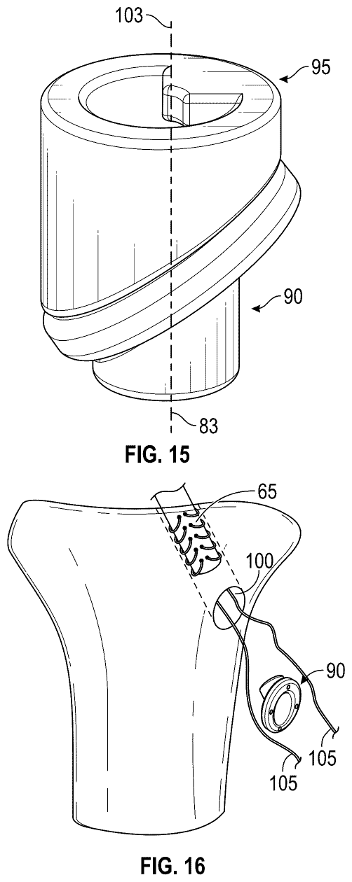

FIG. 14A is a perspective view of the RCBL 90 and the tensioning block 95 prior to engagement according to an embodiment of the present disclosure. FIG. 14B is a perspective view of the RCBL 90 and the tensioning block 95 prior to engagement according to an embodiment of the present disclosure. FIG. 15 is a perspective view of the RCBL 90 and the tensioning block 95 after engagement of the two components according to an embodiment of the present disclosure.

The tensioning block 95 may further include a central axis 103. An angle between the distal surface 102 and the proximal surface 97 of the tensioning block 95 may be generally equal to the axis angle 81 of the RCBL 90. Additionally, with the tensioning block 95 engaged with the RCBL 90, the central axis 103 may be generally colinear with the second axis 83 of the RCBL 90. With the tensioning block 95 engaged with the RCBL 90, the locating apertures 93 and the locating bosses 96 may inhibit rotation and translation of the tensioning block 95 relative to the RCBL 90, and translation of the distal surface 102 relative to the proximal end 88.

In an embodiment, the tensioning block 95 may be one of a set of differently-sized tensioning blocks 95, each having a differently sized outside portion 99, and/or a differently sized angle between the distal surface 102 and the proximal surface 97. A tensioning block 95 may be chosen from the set of differently sized tensioning blocks so that the outside portion 99 is generally equal to the first outside portion 85 of a chosen RCBL 90 and the angle between the distal surface 102 and the proximal surface 97 is generally equal to the axis angle of the chosen RCBL.

The implant disclosed herein, including the CBL 10, the locking fastener 50, the washer 55, and/or the RCBL 90, may be fabricated from any of a variety of biocompatible materials, such as, but not limited to: stainless steel, cobalt chrome, titanium and its alloys, nickel titanium alloy, polyetheretherketone (PEEK), carbon fiber reinforced PEEK, porous PEEK, biocompatible polymers, and/or combinations of the foregoing. Additionally, or alternatively, the implants disclosed herein may also encompass a variety of surface treatments or additives to encourage bony attachment, including but not limited to: porous coatings, hydroxyapatite, TCP (tricalcium phosphate), anti-microbial additives, analgesics, anti-inflammatories, PRP (platelet-rich plasma), BMPs (bone morphogenic proteins), PMA (phorbol myristate acetate) material, bone growth promoting material, PLLA (poly-L-lactide), PGA (polyglycolide), TCP (tricalcium phosphate), demineralized bone, cancellous bone chips, etc. Any implant disclosed herein may include a subtractive etching process that may result in microscopic cuts in a surface of an implant. A subtractive etching process may promote in growth of bony material into an implant. Any implant disclosed herein may include a radiographic marker for imaging purposes. Any implant disclosed herein may be colored, coded or otherwise marked to make it easier for the surgeon to identify the type and size of the implant.

Any implant or instrument disclosed herein may be manufactured through additive manufacturing techniques, such as 3D printing, subtractive manufacturing techniques, such as machining, or other manufacturing techniques known in the art, such as injection molding and/or forging. Any implant disclosed herein may be manufactured with a lattice structure or a combination of generally solid portions and portions with a lattice structure. A lattice structure may be included on a bottom surface, a top surface and/or through an implant. A lattice structure may be configured to promote bone in-growth.

The instruments disclosed herein, including the spiked obturator 20, the dilator tube 30, the suture lock holder 40, the fastener driver, the tensioning block 95, and/or the tensioning tool 115, may be may be fabricated from any of a variety of biocompatible materials, such as, but not limited to: stainless steel, aluminum, titanium and its alloys, polyetheretherketone (PEEK), carbon fiber reinforced PEEK, polycarbonate, polyamide, Radel, Ultem, ABS, other biocompatible polymers, and/or combinations of the foregoing.

The instrument and/or implants described herein may be manufactured through additive manufacturing techniques, such as 3D printing, subtractive manufacturing techniques, such as machining, or other manufacturing techniques known in the art, such as injection molding and/or forging.

FIG. 16 is a perspective view of a bone tunnel 100 prepared for placement of the RCBL 90 according to an embodiment of the present disclosure. A revision ligament graft reconstruction procedure may include removal of a previous ligament graft and/or implant, grafting a previous bone tunnel, identification of a preferred bone tunnel trajectory, and/or creation of a bone tunnel.

The sutures 105 may be one or more sutures, and/or one or more suture tapes. The sutures 105 may extend from a ligament graft 110. The ligament graft 110 may be passed through a distal femur and may be fixed on an external portion of the femur using the suture fixation system 5 as described above and/or another preferred fixation method. The sutures may be secured to the ligament graft using any suturing technique known in the art, such as Krackow, McKeon's Double Krackow, Wilson's Double Krackow, Ostrander's Modified Krackow, Giftbox-modified Krackow, Kessler, or Bunnell. The axis angle 81 of the RCBL 90 may generally correspond to an angle of a longitudinal axis of the bone tunnel 100 relative to a surface of the bone so that the ledge portion 86 of the RCBL 90 may generally sit flush with the surface of the bone.

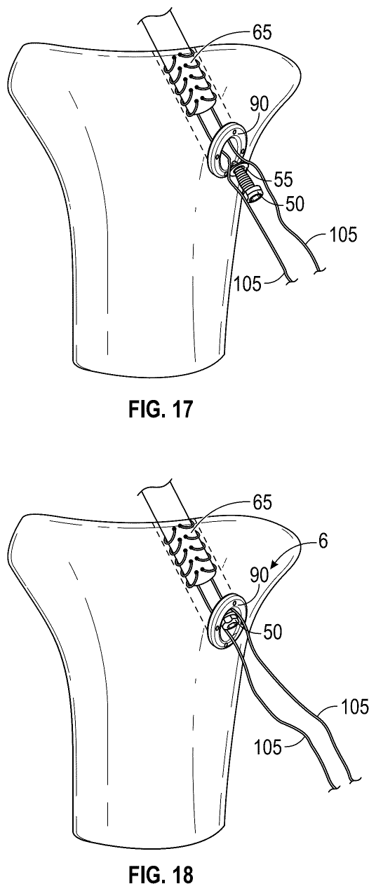

FIG. 17 is a perspective view of the bone tunnel 100 after placement of the RCBL 90 and prior to final placement of the washer 55 and the locking fastener 50 according to an embodiment of the present disclosure. The sutures 105 may be passed through the apertures 92 and the RCBL 90 may be fully inserted into the bone tunnel 100 so that the ledge portion 86 may contact the surface of the bone. The locking fastener 50 may be received with the washer 55 prior to engagement of the locking fastener 50 with the RCBL 90.

FIG. 18 is a perspective view of the bone tunnel 100 after placement of the RCBL 90 and prior to final locking of the washer 55 and the locking fastener of 50 and with passage of sutures through the suture apertures 92 of the RCBL 90 according to an embodiment of the present disclosure. The locking fastener 50 may be preliminarily engaged with the RCBL 90 so that the sutures 105 may freely slide within the suture apertures 92.

FIG. 19 is a perspective view of the bone tunnel 100 after placement of the RCBL 90 and the tensioning block 95 with sutures 105 tied around a tensioning tool 115 according to an embodiment of the present disclosure. The tensioning block 95 may engage the RCBL 90. The sutures 105 may be passed through the central aperture 98 of the tensioning block 95. The sutures 105 may then be knotted so that a tensioning tool 115 may be introduced between the knotted sutures and the proximal surface 97 of the tensioning block 95. The tensioning tool 115 may be used to pull the sutures 105 thereby tensioning the ligament graft 110 a desired amount.

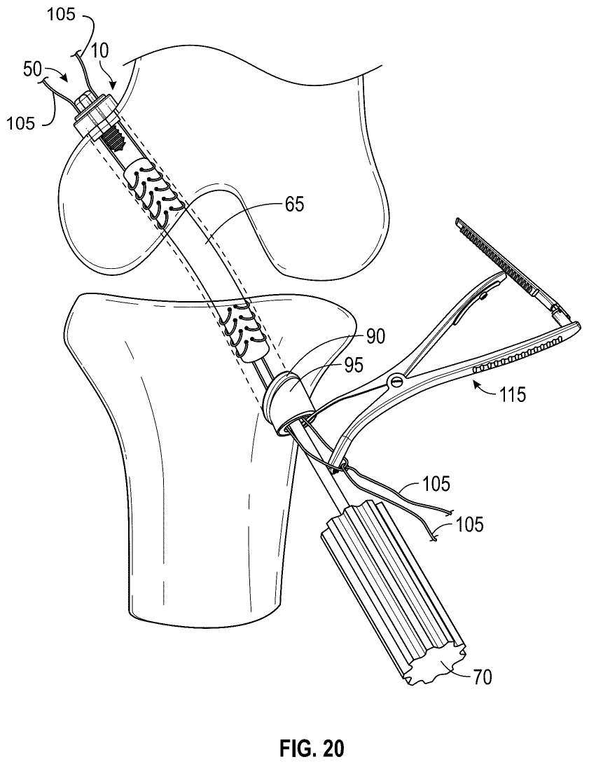

FIG. 20 is a perspective view of the fastener driver 70, the bone tunnel 100 after placement of the RCBL 90 and the tensioning block 95 with sutures 105 tied around the tensioning tool 115 according to an embodiment of the present disclosure. With the tensioning tool 115 pulling the sutures 105 and tensioning the ligament graft 110 a desired amount, the fastener driver 70 may be introduced into the central aperture 98 of the tensioning block 95 and may engage the drive feature 51 of the locking fastener 50. The locking fastener 50 may be tightened to secure the sutures 105 to the RCBL 90, thereby maintaining the tension on the ligament graft 110.

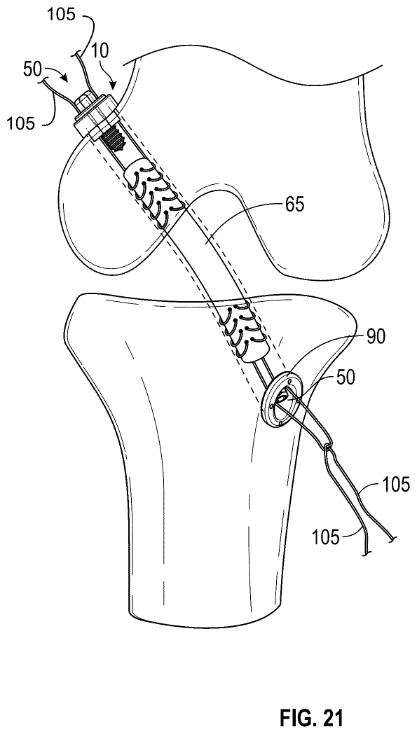

FIG. 21 is a perspective view of the bone tunnel 100 after placement of the RCBL 90 and after tensioning of the sutures 105 and final tightening of the locking fastener 50 and after removal of the fastener driver 70 according to an embodiment of the present disclosure. After final tightening of the locking fastener 50, the tensioning tool 115 may be removed from the sutures 105 and the tensioning block 95. With the locking fastener 50 securing the sutures 105 to the RCBL 90 and inhibiting the sutures 105 from sliding relative to the RCBL 90, the ends of the sutures 105 may be tied over the locking fastener 50, cut, and/or fixed using a secondary fixation technique known in the art.

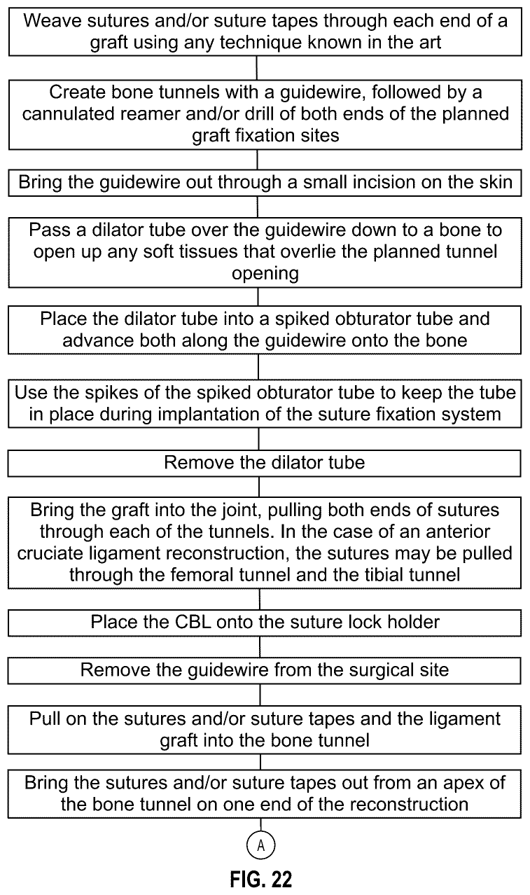

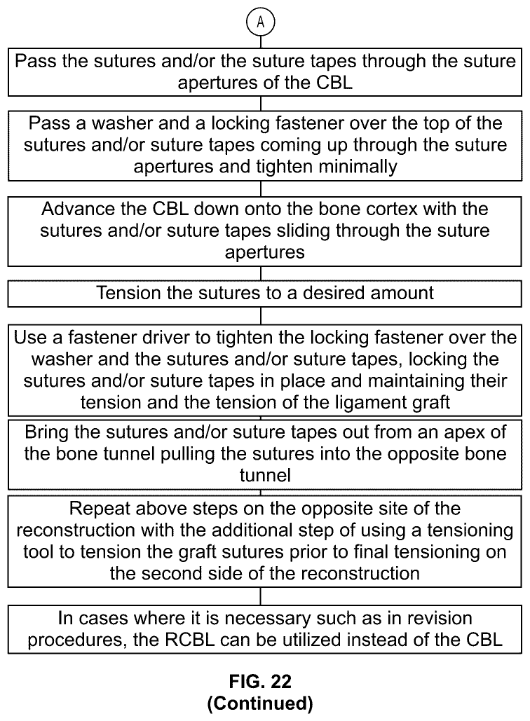

FIG. 22 is an exemplary method for ligament reconstruction using a suture fixation system according to an embodiment of the present disclosure. An exemplary method for ligament reconstruction using a suture fixation system may include the following steps:
1. Weave sutures and/or suture tapes through each end of a graft using any technique known in the art.
2. Create bone tunnels with a guidewire, followed by a cannulated reamer and/or drill of both ends of the planned graft fixation sites.
3. Bring the guidewire out through a small incision on the skin.
4. Pass a dilator tube over the guidewire down to a bone to open up any soft tissues that overlie the planned tunnel opening.
5. Place the dilator tube into a spiked obturator tube and advance both along the guidewire onto the bone.
6. Use the spikes of the spiked obturator tube to keep the tube in place during implantation of the suture fixation system.
7. Remove the dilator tube.
8. Bring the graft into the joint, pulling both ends of sutures through each of the tunnels. In the case of an anterior cruciate ligament reconstruction, the sutures may be pulled through the femoral tunnel and the tibial tunnel.
9. Place the CBL onto the suture lock holder.
10. Remove the guidewire from the surgical site.
11. Pull on the sutures and/or suture tapes and the ligament graft into the bone tunnel.
12. Bring the sutures and/or suture tapes out from an apex of the bone tunnel on one end of the reconstruction.
13. Pass the sutures and/or the suture tapes through the suture apertures of the CBL.
14. Pass a washer and a locking fastener over the top of the sutures and/or suture tapes coming up through the suture apertures and tighten minimally.
15. Advance the CBL down onto the bone cortex with the sutures and/or suture tapes sliding through the suture apertures.
16. Tension the sutures to a desired amount.
17. Use a fastener driver to tighten the locking fastener over the washer and the sutures and/or suture tapes, locking the sutures and/or suture tapes in place and maintaining their tension and the tension of the ligament graft.
   a. Note: the protrusions of the suture lock holder may resist the torque from the fastener driver while holding the CBL in a stable position over the bone.
18. Repeat step 12, pulling the sutures into the opposite bone tunnel.
19. Repeat steps 13-18 on the opposite site of the reconstruction with the additional step of using a tensioning tool to tension the graft sutures prior to final tensioning on the second side of the reconstruction.
20. In cases where it is necessary such as in revision procedures, the RCBL can be utilized instead of the CBL.

Those of skill in the art will recognize that this is only one of many potential methods that may be used for suture fixation systems for ligament reconstruction. In alternative embodiments, different methods may be used for suture fixation systems for ligament reconstruction other than the methods described above. Further, the methods set forth above may be used to secure other suture fixation systems besides those specifically disclosed herein.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, instruments, and methods disclosed herein.

What is claimed is:

1. A suture fixation system for ligament reconstruction configured to tension and secure a ligament graft to a bone using one or more sutures, the system comprising:
a suture lock comprising:
a proximal end comprising a first portion;
a distal end comprising a second portion that is smaller than the first portion and configured to be received in a bone tunnel;
a ledge portion configured to inhibit the first portion from entering the bone tunnel; and
one or more suture apertures extending from the distal end to the proximal end and configured to allow the one or more sutures to pass through the suture lock; and
a fastener comprising a fastener head and configured to engage the suture lock;
wherein, with the one or more sutures applying tension to the ligament graft, the fastener is configured to securably pinch the one or more sutures between the fastener head and the suture lock to inhibit sliding of the one or more sutures relative to the suture lock to maintain the tension of the ligament graft.

2. The system of claim 1, further comprising a washer comprising a central aperture configured to receive the fastener and a locking portion, wherein the suture lock further comprises a proximal recess, and, with the locking portion engaged in the proximal recess, the washer is configured to inhibit loosening of the fastener relative to the suture lock.

3. The system of claim 1, wherein the system further comprises a suture lock holder comprising a second distal end, wherein the second distal end is configured to receive the suture lock and the sutures, and wherein the first portion comprises a plurality of notches and the second distal end comprises a plurality of protrusions configured to be received in the plurality of notches, and the suture lock holder is configured to removably retain the suture lock and facilitate placement of the suture lock within the bone tunnel.

4. The system of claim 3, wherein the suture lock holder further comprises an inside portion configured to allow the one or more sutures to pass through the inside portion with the suture lock retained in the suture lock holder.

5. The system of claim 1, wherein the suture lock further comprises a first threaded portion and the fastener further comprises a second threaded portion, wherein the second threaded portion is configured to threadably engage the first threaded portion.

6. The system of claim 1, wherein the second portion is angularly offset from the first portion so that the second portion aligns with the bone tunnel and the ledge portion aligns with a bone surface proximate the bone tunnel.

7. The system of claim 6, further comprising a tensioning block comprising a first surface configured to engage the proximal end of the suture lock and a second surface angularly offset from the first surface, wherein, with the tensioning block engaged with the suture lock, a first longitudinal axis of the tensioning block is parallel with a second longitudinal axis of the second portion.

8. A suture fixation system for ligament reconstruction configured to tension and secure a ligament graft to a bone using one or more sutures, the system comprising:
a suture lock comprising:
a proximal end comprising a first portion;
a distal end opposite the proximal end comprising a second portion; and
one or more suture apertures extending from the distal end to the proximal end and configured to allow the one or more sutures to pass through the suture lock;
a fastener comprising a fastener head, wherein the fastener is configured to engage the suture lock; and
a suture lock holder comprising a second distal end, wherein the second distal end is configured to receive the suture lock and the sutures;
wherein:
the first portion comprises a plurality of notches;
the second distal end comprises a plurality of protrusions configured to be received in the plurality of notches;
the suture lock holder is configured to removably retain the suture lock and facilitate placement of the suture lock within a bone tunnel; and
with the one or more sutures applying tension to the ligament graft, the fastener is configured to securably pinch the sutures between the fastener and the suture lock to inhibit sliding of the one or more sutures relative to the suture lock and maintain the tension of the ligament graft.

9. The system of claim 8, further comprising a washer comprising a central aperture configured to receive the fastener and a locking portion, and, wherein the washer is configured to inhibit loosening of the fastener relative to the suture lock and securably pinch the sutures between the locking portion and the suture lock.

10. The system of claim 9, wherein the suture lock holder further comprises an inside portion configured to allow the one or more sutures to pass through the inside portion with the suture lock retained in the suture lock holder.

11. The system of claim 8, wherein the second portion is angularly offset from the first portion so that the second portion aligns with the bone tunnel and the first portion aligns with a bone surface proximate the bone tunnel.

12. The system of claim 11, further comprising a tensioning block comprising a first surface configured to engage the proximal end of the suture lock and a second surface angularly offset from the first surface, wherein, with the tensioning block engaged with the suture lock, a first longitudinal axis of the tensioning block is parallel with a second longitudinal axis of the second portion.

13. The system of claim 12, wherein the first surface of the tensioning block comprises a plurality of locating bosses, the first portion of the suture lock further comprises a plurality of locating apertures, and with the tensioning block engaged with the suture lock, the plurality of locating bosses and the plurality of locating apertures inhibit rotation and translation of the tensioning block relative to the suture lock.

14. A suture fixation system for ligament reconstruction configured to tension and secure a ligament graft to a bone using one or more sutures, the system comprising:
a suture lock comprising:
a first portion comprising a proximal recess and a first longitudinal axis;
a second portion that is smaller than the first portion and configured to be received in a bone tunnel and comprising a second longitudinal axis that is not parallel to the first longitudinal axis;
a ledge portion configured to inhibit the first portion from entering the bone tunnel; and
one or more suture apertures extending through the second portion to the proximal recess and configured to allow the one or more sutures to pass through the suture lock;
a fastener comprising a fastener head, wherein the fastener is configured to engage the suture lock; and
a washer configured to be received in the proximal recess and comprising a central aperture configured to receive the fastener;
wherein:
with the one or more sutures applying tension to the ligament graft, the fastener securably pinches the one or more sutures between the washer and the suture lock to inhibit sliding of the one or more sutures relative to the suture lock and maintain the tension of the ligament graft.

15. The system of claim 14, wherein the suture lock comprises an axis angle between the first longitudinal axis and the second longitudinal axis and the axis angle is between 30 degrees and 70 degrees.

16. The system of claim 14, wherein the washer comprises an outside portion comprising a locking portion, wherein the locking portion is configured to securably pinch the one or more sutures between the washer and the suture lock to inhibit the sliding of the one or more sutures relative to the suture lock and maintain the tension of the ligament graft.

17. The system of claim 14, wherein the suture lock further comprises a first threaded portion and the fastener further comprises a second threaded portion, wherein the second threaded portion is configured to threadably engage the first threaded portion.

18. The system of claim 14, further comprising a tensioning block comprising a first surface configured to engage the first portion of the suture lock and a second surface angularly offset from the first surface, wherein, with the tensioning block engaged with the suture lock, a third longitudinal axis of the tensioning block is parallel with the second longitudinal axis.

19. The system of claim 18, wherein the first surface of the tensioning block comprises a plurality of locating bosses, the first portion of the suture lock further comprises a plurality of locating apertures, and, with the tensioning block engaged with the suture lock, the plurality of locating bosses and the plurality of locating apertures inhibit rotation and translation of the tensioning block relative to the suture lock.

20. The system of claim 18, wherein the tensioning block further comprises a second central aperture, the fastener head comprises a drive feature, and, with the tensioning block engaged with the suture lock, a fastener driver may engage the drive feature through the second central aperture to secure the fastener to the suture lock.
